(19) United States
(12) Patent Application Publication
(10) Pub. No.: US 2022/0084636 A1
(43) Pub. Date: Mar. 17, 2022

(54) MACHINE LEARNING ANALYSIS FOR METABOLOMICS CLASSIFICATION AND BIOMARKER DISCOVERY

(71) Applicant: THE BOARD OF TRUSTEES OF THE LELAND STANFORD JUNIOR UNIVERSITY, Stanford, CA (US)

(72) Inventors: Catherine HOGAN, Stanford, CA (US); Pranav Rajpurkar, Stanford, CA (US); Benjamin Alan Pinsky, San Francisco, CA (US); Anthony T. Le, San Jose, CA (US)

(73) Assignee: THE BOARD OF TRUSTEES OF THE LELAND STANFORD JUNIOR UNIVERSITY, Stanford, CA (US)

(21) Appl. No.: 17/475,271

(22) Filed: Sep. 14, 2021

Related U.S. Application Data
(60) Provisional application No. 63/078,320, filed on Sep. 14, 2020.

(51) Int. Cl. G16H 10/40; G16H 20/00; G16B 40/10; G16B 40/20; G06N 20/20

(52) U.S. Cl. CPC G16H 10/40; G16H 20/00; G06N 20/20; G16B 40/20; G16B 40/10

(57) ABSTRACT

The present invention relates to systems, methods and devices for metabolomic-based classification of biological samples, and interpretation methods for biomarker discovery.

### BACKGROUND

Metabolomics involves the study of the metabolism and metabolites in an organism. Metabolome studies review the qualitative and quantitative characterization of small molecules with changes appearing in organisms in response to a variety of endogenous and exogenous stimuli. The metabolome is unique, dynamic and concerns phenotype. Metabolomics, for example, is able to link both gene and environmental interactions. It represents genomic output and environmental input. In recent years, metabolomics approaches have been applied to various fields since it can detect subtle changes in a large dataset with comprehensive metabolite measurements. The metabolites present in biological systems include endogenously derived biochemicals. In general, metabolomics is a valuable tool in different disciplines such as drug discovery, biomarker research, studies of diseases, and metabolic pathways confirmation. It involves both the identification of endogenous substances in different biological samples as well as the statistical analysis of differences between two or more conditions.

In practice, metabolomics is a diagnostic approach that can be performed by either looking for all compounds present in the sample (untargeted approach), or by limiting analysis to selected compounds only (targeted approach). Samples are run and data are generated for accurate mass and retention time for each compound. In the untargeted approach, the number of compounds per sample can reach over 20,000. However, there is no commercial software package currently available that can quantitatively analyze the data to generate test performance data. Available methods involve intense manual inputs and manipulations and therefore are fraught with difficulties, subjective interpretations, low reproducibility, non-comprehensive, and are time-consuming.

The present innovations address these and other needs in the art.

### SUMMARY

As provided herein a variety of systems and methods are contemplated herein.

According to frequent embodiments, a computer-implemented method is provided involving generating or receiving a plurality of metabolite feature data using a processed sample from a subject with an unknown or uncertain diagnosis or prognosis; applying selective metabolite features to the plurality of metabolite feature data to create a new data output; and generating a diagnostic or prognostic indication for the subject based on the new data output, wherein the selective metabolite features are obtained by subjecting a plurality of corresponding metabolite feature data to a LightGBM machine learning model and a random forest (RF) machine learning model to generate classified corresponding metabolite feature data, the classified corresponding metabolite feature data comprising the plurality of corresponding metabolite feature data organized based on a ranking of a plurality of mass spectrometry identified features; and identifying a subset of the classified corresponding metabolite features as the selective metabolite features for a disorder using a SHapley Additive exPlanations (SHAP) method. In certain embodiments, the selective metabolites features are selected from one or more selective metabolites features listed in Table S3, FIG. 3A and/or FIG. 4.

In often included embodiments, each of the plurality of metabolite feature data is obtained using a patient sample having a known diagnostic or prognostic status.

Also often, the processed sample is obtained from eluting and processing a raw subject sample by liquid chromatography, and wherein the plurality of metabolite feature data is obtained by subjecting the processed sample to mass spectroscopy. Frequently, the liquid chromatography is two column in-line liquid chromatography comprising reverse phase and ion exchange chromatography. In such embodiments, the in-line chromatography comprises reverse phase chromatography followed by ion exchange chromatography.

In frequent embodiments, the method involves a process whereby the eluting and processing comprises ultrafiltration of the raw subject sample, and the raw subject sample comprises a nasopharyngeal swap in transport medium. Often the raw subject sample comprises any of the sample types contemplated herein and the raw subject sample is subjected to processing such that the sample can be analyzed by mass spectroscopy.

In frequent embodiments, the selective metabolite features comprises one or more features. Often, the selective metabolite features comprises three or more features. Often, the selective metabolite features comprises 3, 5 or 7 features. Also often, the selective metabolite features comprise between 1 to 20 features. Also often, the selective metabolite features comprise between 3 to 20 features. Also often, the selective metabolite features comprise between 5 to 20 features. Also often, the selective metabolite features comprise between 7 to 20 features. Also often, the selective metabolite features comprise between 1 to 7 features. Also often, the selective metabolite features comprise between 1 to 10 features. Also often, the selective metabolite features comprise between 1 to 15 features. Also often, the selective metabolite features comprise between 3 to 7 features. Also often, the selective metabolite features comprise between 3 to 5 features. Also often, the selective metabolite features comprise between 1 to 5 features. Also often, the selective metabolite features comprise between 1 to 3 features.

In certain embodiments, pyroglutamic acid is one of the selective metabolite features and the diagnostic or prognostic indication relates to influenza or infection by a respiratory virus. Often, the diagnostic or prognostic indication relates to influenza H1N1, influenza H3 and/or influenza B. In certain embodiments, the selective metabolites features are selected from one or more selective metabolites features listed in Table S3, FIG. 3A and/or FIG. 4.

In often included embodiments, the diagnostic or prognostic indication relates to an infectious disease state, a cancer state, graft rejection state, a blood disorder, a soft tissue disorder, or an autoimmune disease state.

The presently described embodiments often comprise methods is conducted at a point-of-care facility such as a doctor office, hospital, clinic, urgent care facility, or other similar location. Frequently, the method is conducted at the point-of-care of the subject and the mass spectroscopy is conducted on site, for example, using a portable mass spectroscopy device or other device.

In often included embodiments, the generated diagnostic or prognostic indication for the subject based on the new data output is utilized in conjunction with clinical data in a diagnosis of or prognosis for the subject.

Also often, the subject is identified as eligible for treatment based on the diagnostic or prognostic indication without associated genetic or molecular data obtained from a raw sample corresponding to the processed sample. In such embodiments, often the treatment comprises treatment for influenza, another infectious respiratory disease, cancer, graft rejection, a blood disorder, a soft tissue disorder, and/or autoimmune disease.

Also provided in frequent embodiments described herein is a method of processing a biological sample from a subject for metabolomics classification involving either (i) eluting and processing the biological sample by liquid chromatography to create a processed sample and subjecting the biological sample to mass spectrometry to obtain a plurality of metabolite feature data, or (ii) obtaining the plurality of metabolite feature data from a preprocessed sample; subjecting the plurality of metabolite feature data to a LightGBM machine learning model and a random forest (RF) machine learning model to generate classified metabolite feature data, the classified metabolite feature data comprising the plurality of metabolite feature data organized based on a ranking of a plurality of mass spectrometry identified features; and identifying a subset of the classified metabolite features as selective metabolite features for a disorder using a SHapley Additive exPlanations method. In certain embodiments, the selective metabolites features are selected from one or more selective metabolites features listed in Table S3, FIG. 3A and/or FIG. 4.

Often the classified metabolite features are applied to a sample or series of samples, including an agent-treated sample or samples, in a process of biomarker discovery or analysis.

According often included embodiments, a method of processing a biological sample from a subject for metabolomics classification is provided, comprising: obtaining the biological sample from a subject suspected of being afflicted with a disorder; subjecting the biological sample to mass spectrometry to obtain a plurality of metabolite feature data; subjecting the plurality of metabolite feature data to a LightGBM machine learning model and a random forest (RF) machine learning model to generate classified metabolite feature data, the classified metabolite feature data comprising the plurality of metabolite feature data organized based on a ranking of a plurality of mass spectrometry identified features; identifying a subset of the classified metabolite features as selective metabolite features for the disorder using a t method; obtaining a test biological sample from the subject or a second subject; subjecting the test biological sample to mass spectrometry to obtain the plurality of metabolite feature data; and analyzing the plurality of metabolite feature data for the selective metabolite features for the disorder and identifying the sample regarding a status of the sample for the disorder; and administering a treatment for the disorder to the subject or the second subject based on the identifying the sample as having a positive status for the disorder. In certain embodiments, the selective metabolites features are selected from one or more selective metabolites features listed in Table S3, FIG. 3A and/or FIG. 4.

In certain frequent embodiments, the disorder is: influenza and the treatment is an influenza treatment; an infectious disease and the treatment is specific for that infectious disease; a blood disorder the treatment is specific for the blood disorder; and/or a soft tissue disorder and the treatment is specific for the soft tissue disorder. In certain embodiments, the disorder is unknown prior to subjecting the sample to the present methods and the disorder is identified and a treatment is identified and optionally administered for the identified disorder according to the present systems and methods.

In certain frequent embodiments the methods involve evaluation of the plurality of metabolite feature data apart from obtaining the sample and/or subjecting the biological sample to mass spectrometry.

In certain frequent embodiments, the mass spectrometry comprises liquid chromatography quadrupole time-of-flight mass spectrometry.

In certain frequent embodiments, the subject or the second subject each together or independently comprise a plurality of subjects.

In certain embodiments, the present systems and methods are utilized to analyze a set of data comprising multiple subjects. In certain embodiments the subject or subjects are suspected of separately diagnosed as having one or more specific disorders and metabolomic data of the samples from the subjects are evaluated according to the present methods to identify or confirm metabolomic biomarkers indicative of the one or more specific disorders. In certain related embodiments, the disorder status of the subject or subjects is unknown and the present methods and systems are utilized to diagnose or confirm a diagnosis concerning the disorder status for the subject or subjects.

Systems operable for conducting and/or adapted to conduct the methods described herein are frequently contemplated embodiments.

Also provided in often included embodiments are devices adapted to conduct the methods described herein. Frequently such devices include a processor and are operably connected with computer executable code, memory and data storage to support the method in an onboard computer or a remote computer. Often, a remote computer is utilized to conduct the relevant statistical analyses on the data generated from the subject sample. Frequently, an onboard computer is utilized to conduct the relevant statistical analyses on the data generated from the subject sample. Also often, the devices are adapted to perform sample purification, liquid chromatography and/or mass spectroscopy.

It is understood that the present systems often include one or more processors operably connected with a tangible storage medium, software, data inputs/outputs and/or connections, and/or often a portal or interface for operating the system. The methods described herein often operate utilizing such hardware and software. Algorithms and machine learning models described herein are often stored on the tangible storage medium and/or employed as an operable component of the software.

These and other embodiments, features, and advantages are apparent to those skilled in the art when taken with reference to the following more detailed description of various exemplary embodiments of the present disclosure in conjunction with the accompanying drawings.

### BRIEF DESCRIPTION OF THE DRAWINGS

The skilled person in the art will understand that the drawings, described below, are for illustration purposes only. The drawings are incorporated in and constitute a part of this specification.

FIG. 1 provides a conceptual diagram of the study. The phases of data collection, model development, and interpretation are illustrated. LC/Q-TOF: liquid chromatography quadrupole time-of-flight; LC-MS/MS: liquid chromatography-mass spectrometry; RF: random forests; ROC: receiver operating characteristic curve; SHAP: Shapley additive explanation.

FIG. 2A depicts ROC curves comparing the performance of the machine learning models (RF, LightGBM) with the traditional linear models (Lasso, Ridge) on the test set; bracketed values are 95% AUC confidence intervals calculated from a normal fit of the curves. AUC: area under the receiver operating characteristic curve; ImmunoC: immunocompromised; Ped: pediatric; RF: random forests; ROC: receiver operating characteristic curve.

FIG. 2B depicts ROC curves of comparing LightGBM's performance on the test set stratified by pediatrics. 95% confidence intervals are shown in brackets.

FIG. 2C depicts ROC curves of comparing LightGBM's performance on the test set stratified by immunocompromised. 95% confidence intervals are shown in brackets.

FIG. 2D depicts ROC curves comparing LightGBM's performance on the prospective test set; bracketed values are 95% AUC confidence intervals calculated from a normal fit of the curves.

FIG. 3A depicts top 20 ion features by percentage importance using the SHAP method. Ion features are identified by accurate mass @ retention time, and colors indicate the association between feature value and positive influenza classification. For example, low values of 84.0447@0.81 are indicative of positive classification, while the relative value of 106.0865@10.34 does not have a clear interpretation, despite being an important feature. AUC: area under the receiver operating characteristic curve; m/z: mass over charge ratio; RT: retention time

FIG. 3B depicts AUC and 95% confidence interval of parsimonious decision tree models as a function of number of features used for training in the retrospective discovery (blue) and prospective (green) cohorts.

FID. 3C depicts an example decision tree model trained using only the top feature and a maximum depth of 1 that has an AUC of greater than 0.9 on the test set.

FIG. 4 depicts a heatmap of nasopharyngeal metabolites. This heatmap was generated from metabolomics analysis of nasopharyngeal samples from children and adults with and without influenza infection, clustered by correlation distance and average linkage. The accurate mass and retention time (accurate mass @ retention time) are listed for each compound on the right, the hierarchical cluster tree appears on the left, and the influenza virus type or subtype is listed at the bottom.

FIG. 5 depicts an Area under the curve (AUC) data with viral transport medium subtraction. This model subtracted the mean viral transport medium (VTM) data to assess the impact of background matrix in the analysis. The estimates presented are similar to those without VTM subtraction.

### DETAILED DESCRIPTION

For clarity of disclosure, and not by way of limitation, the detailed description of the invention is divided into the subsections that follow.

Unless otherwise defined herein, scientific and technical terms used in connection with the present application shall have the meanings that are commonly understood by those of ordinary skill in the art to which this disclosure belongs. This disclosure is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

All patents, applications, published applications and other publications referred to herein are incorporated by reference in their entirety.

The terminology used in the description of the various described embodiments herein is for the purpose of describing particular embodiments only and is not intended to be limiting. As used in the description of the various described embodiments and the appended claims, the singular forms “a”, “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, “subject” often refers to an animal, including, but not limited to, a primate (e.g., human). The terms “subject” and “patient” are used interchangeably herein.

As used herein, “sample” refers to any substance containing or presumed to contain a marker or feature of interest for investigation. The term “sample” thus includes a cell, organism, tissue, fluid, or substance or fragment thereof (including proteins, polypeptides, or nucleic acids), including but not limited to, for example, blood, plasma, serum, spinal fluid, lymph fluid, synovial fluid, urine, tears, stool, external secretions of the skin, respiratory, intestinal and genitourinary tracts, saliva, blood cells, tumors, organs, tissue, samples of cell culture constituents, natural isolates (such as drinking water, seawater, solid materials), microbial specimens, cell lines, and plant cells. A “tissue sample” refers to a sample having or obtained from a tissue of a subject, including homogenized, disassociated, otherwise processed samples, cellular cultures thereof, and fractions or expression products thereof. The sample often requires processing to enable mass spectrometry or another relevant analysis contemplated herein, and therefore the term “sample” is intended to refer to the sample before or after such processing. For example, a sample may be a purified and separated nasopharyngeal sample using filtration/ultrafiltration and/or liquid chromatography. A variety of techniques known to those of ordinary skill in the art may be used for this purpose.

As used herein, “treatment” means any manner in which the symptoms of a condition, disorder or disease are ameliorated or otherwise beneficially altered. Treatment also encompasses any pharmaceutical use of the compositions herein.

As used herein, the terms “detect,” “detecting,” or “detection” may describe either the general act of discovering or discerning or the specific observation of a molecule or composition, whether directly or indirectly labeled with a detectable label.

As used herein “diagnosis” refers to the ability of a test to determine, yes or no, if a patient is positive for a disease state.

As used herein “prognosis” refers to the ability of a test to determine how aggressive of indolent a disease state is, in part by predicting specific pathology findings related to the progression of a disease.

As used herein, “computer executable” includes instructions and data which, when executed at one or more processors, cause a general-purpose computing system, special-purpose computing system, or special-purpose processing device to perform a certain function or group of functions. Computer-executable instructions may be, for example, binaries, intermediate format instructions such as assembly language, or even source code. Computer-executable instructions, therefore, include any software, including low level software written in machine code, higher level software such as application software and any combination thereof. In this regard, the system components can manage resources and provide services for the system functionality. Any other variations and combinations thereof are contemplated with embodiments of the present disclosure.

As used herein, “operably connected” refers to two or more components, such as two or more modules, are directly or indirectly connected to permit or perform a function for which at least one of the components/modules is specified.

As noted, according to the presently described methods, two major kinds of metabolomic analyses are often applied—targeted and untargeted. Targeted analysis focuses on a known number of defined metabolites, but untargeted metabolomics or discovery metabolomics aims to capture all metabolomic information in a sample. In the latter, typically features of interest are filtered after data acquisition applying different statistical methods followed by their identification. If reference material is not available for the metabolites of interest a comparison between groups or conditions is performed on the basis of relative abundances of metabolites. Reproducible measurements are therefore required for reliable data processing and analysis. Typically, samples of one metabolome experiment are measured within the same batch in order to avoid bias caused by sampling, storage or time variations in instrument performance. Metabolomics platforms generate a large amount of data that is also complex, therefore highlighting the need for appropriate data processing tools that allow the uniform and normalized preparation of chromatographic and spectral data for data analysis.

Different kinds of statistical tests are described herein and have been performed for data interpretation. Univariate tests (e.g., t-test; ANOVA) compare the intensities of single features between different groups. Nevertheless, the requirement for repeated accounting for and analysis of many variables in metabolomic studies increases the risk for detection of false positives. This is often accounted for by applying false discovery corrections. One widely used unsupervised multivariate technique is principal component analysis (PCA). PCA projects the maximum variance of a multi-dimensional space in principal components and summarizes the data set in a limited number of components. PCA is mainly used as an exploratory technique since it is unsupervised and therefore does not account for class-based separations.

From an analytical perspective, untargeted analyses are provided as advantageous methods according to the present disclosure for identifying unknown metabolites and pathways. While historically such approaches are broadly applicable across a large range of metabolites, they have lacked sensitivity for metabolites present in very small concentrations. Up to now, however, targeted studies considering results from untargeted analysis have been rarely performed.

The present disclosure is focused on the analysis of metabolomic data generated by the variety of methods and instruments in the art that provide access to such datapoints and is less concerned with the method of generation of these metabolomic data, provided appropriate normalization of the data is performed. For example, the present systems and methods provide a toolkit necessary to enable robust biomarker discovery and analysis, regardless of clinical application. Suspect infectious diseases, cancer diagnosis and prognosis, and detection of organ rejection are examples of areas where the presently described systems and methods may be employed.

Robust, reproducible and comprehensive metabolomic-based methods and systems for classification of infection status, and an interpretation method for biomarker discovery are provided herein. These methods and systems can be applied broadly for a variety of infections or conditions that are capable of assessment by metabolomics. Overall, a universal metabolomics classification analysis and biomarker discovery analysis pipeline is provided with the presently described systems and methods.

For example, the presently contemplated methods and systems provide not only the feature importance, but also the direction of the difference (relative abundance of the differentiating compound). Furthermore, these methods and systems provide the necessary infrastructure to automate potential biomarker identification. Utilizing machine learning, the present methods and systems are more powerful than the use of PCA, which is the current state of the art.

In particular, as detailed herein, machine learning methods have been developed for the biomarker determination based on the metabolic profile of a sample. As the term is used herein, machine learning refers to a class of techniques that uses data to learn a model that maps an input (the metabolic profile of a sample) to its associated output (the biomarker identification of the sample) and uses this learned model on new inputs (the metabolic profiles of new samples) to make predictions of new outputs (biomarker identification in new samples). Machine learning systems and methods contemplated herein are robust and low to no manual filtering of raw data prior to data export. While not intending to be bound by any specific theory of operation, it has been determined that the machine learning systems and methods described herein adjust to and handle true signal vs. noise cleaner and with greater efficiency than prior methods. Improvement of the speed of analyses, output of conclusions, a decrease in manual input and improvement of metabolic data processing power of computer systems operating with the presently contemplated systems and methods is provided. As such, without human intervention in the process of analyzing raw data, the present systems and methods improve the operation of statistical analysis software and hardware and provide more accurate, more sensitive, and more specific results, correlations, predictive, and/or prognostic and therapeutic data.

In generating the present methods and systems, the raw data points of mass over charge and retention time for each compound often comprise starting data. These data were divided into separate training data and testing data, with the methods and systems being developed using the training data and tested and adapted using testing data. The primary measure of method and system performance is the area under the receiver operating characteristic curve (AUC), which illustrates, for example, the diagnostic discriminative performance of the contemplated methods and systems. Performance measures for the methods and systems also include sensitivity, specificity, and accuracy at an operating point used to binarize the predictions of the contemplated methods and systems.

The data is generally in the format accurate mass, retention time and abundance. The mass spec instrument parameters will influence these raw results and are at the discretion of the user but will not modify their format for export in the model. The standard processing is required for run alignment and peak picking. Although it can be done if desired, in the most frequent embodiments no further data curation is needed prior to export to pipeline.

To determine the usefulness of capturing non-linear relationships with machine learning models, the modelling approaches using two machine learning methods, gradient boosted decision trees and random forests, were compared with two traditional linear models, Least absolute shrinkage and selection operator (LASSO) and Ridge. These models are variants of Logistic regression, a statistical model that uses the logistic function to model the outcome assuming a linear relationship between the features and the outcome. LASSO makes the same linear assumption but alters the model fitting process to select only a subset of the features for use in the final model rather than using all of them. Unlike LASSO, Ridge will not result in a sparse model, but rather addresses multicollinearity in the features by shrinking the weights assigned to correlated variables. The training and test sets, and the cross-validation strategy were identical across the machine learning models and traditional linear models.

The SHapley Additive exPlanations (SHAP) method was often used to quantify an impact of features on the models. The SHAP method explains, for example, prediction by allocating credit among the input features. In this manner feature credit is calculated using SHAP Values, as the change in the expected value of the model's prediction of improvement for a symptom when a feature is observed versus unknown. To uncover clinically important metabolite features that are globally predictive of the outcome, the SHAP values for features on individual predictions are aggregated and reported along with their averaged absolute Shapley contributions as a percent of the contributions of all the features.

Further a set of parsimonious models were developed that were designed to use a small subset of features identified to be important by the feature importance method. The top k features with highest overall importance to the machine learning models were used; we used k values of 1, 3, 5, and 7. On each of these choices, a single decision tree model was trained using the previously described cross-validation strategy to build the parsimonious model. Maximum depth was restricted to k, and we optimized additional hyperparameters using grid search during cross-validation. We compared the performance of the parsimonious models to the full models. The performance of the models are often evaluated using a reserved test set. The primary measure of model performance that is most frequently used is the area under the receiver operating characteristic curve (AUC), which illustrates the diagnostic discriminative performance of the models. Performance measures for the models also included sensitivity, specificity, and accuracy at a high-sensitivity operating point used to binarize the model predictions. A high-sensitivity operating point is often selected using a training set by aggregating the predictions on the k validation folds, and then picking the threshold that produced a model sensitivity closest to 0.9. To assess the variability in estimates, Wilson score confidence intervals for sensitivity, specificity, and accuracy are provided along with DeLong confidence intervals for AUC.

It has been found that the presently described systems and methods provide higher test performance achieved by a unique fusion of 4 models. Two models are statistical, and two models are ML based. The present methods and systems also automate identification and further analysis of the compounds that are most important in the classification. This aspect saves time and provides accurate, robust, and reproducible results.

In practice, it is contemplated that the presently contemplated systems and methods are applied according to the present disclosure in a variety of different diagnostic and therapeutic contexts and involving a variety of infectious diseases and other disorders where metabolomics data are or can be utilized. In frequent embodiments, systems and methods are contemplated embodying the machine learning analysis systems and methods that are employed to analyze metabolomics data and produce classification performance estimates. Also frequently, systems and methods are contemplated embodying the machine learning analysis systems and methods that are employed to analyze feature importance and determine which compounds to include for targeted metabolomics testing. In addition, in often included embodiments, systems and methods are contemplated embodying the machine learning analysis systems and methods that are employed to analyze feature importance and determine which compounds to include for point-of-care testing.

The presently described systems and methods provide significant advantages compared to existing methods. Firstly, these systems and methods include quantitative results that allow much more accurate estimates of test performance. Secondly, the systems and methods outperform the most commonly used models for metabolomics analysis, i.e., random forests. Furthermore, according to the present systems and methods the analysis of feature importance is more automated, streamlined and comprehensive than current methods. Furthermore, the presently described systems and methods are highly reproducible owing to the use of all data by the algorithm, thus eliminating inter-user variation.

The present described systems and methods can often be used in conjunction with LC/Q-TOF raw data of any of a variety of commercially available instruments.

According to frequent embodiments of the presently described systems and methods, machine learning algorithms of Gradient boosted decision trees and Random Forests are applied to machine learning methods for the task of determining whether a sample is/was positive or negative for a disease or disorder (e.g., influenza) based on the metabolic profile of the sample. It has been discovered by the present inventors that gradient boosted decision trees (GBDT) and Random Forests (RF) are ensemble learning methods that improve upon the performance of decision tree models. It has been observed and documented herein that machine learning approaches of GBDT and RF handle mixes of categorical and continuous covariates, capture nonlinear relationships, and scale well to large amounts of data.

Also according to frequent embodiments of the presently described systems and methods, the SHAP method is/was used to quantify the impact of each feature in a selected metabolic profile on the models. If has been discovered that the method explains prediction by allocating credit among the input features; feature credit is calculated using SHAP Values as the change in the expected value of the model's prediction of improvement for a symptom when a feature is observed versus unknown. According to the present disclosure, to uncover clinically important ion features that are/were globally predictive of the outcome, the SHAP values for a pre-selected set of features (e.g., the top 20 ion features) on individual predictions were aggregated and reported along with their averaged absolute SHAP contributions as a percent of the contributions of all the features. In certain embodiments, fewer or greater than 20 features are contemplated in a similar manner.

Also according to frequent embodiments of the presently described systems and methods, the top features according to the Shapley contributions were/are utilized to develop a set of parsimonious models that were designed to use a small subset of features identified to be important by the feature importance method. The top k features with highest overall importance to the machine learning models were used. In the presently provided example, k values of 1, 3, 5, and 7 were used, though others are contemplated. On each of these choices, a single decision tree model was trained.

The present systems and methods are further illustrated and described by the following examples, provided solely to illustrate the invention by reference to specific embodiments. These examples, while illustrating certain specific aspects of the systems and methods disclosed herein, does not portray the limitations or circumscribe the scope of the present disclosure.

Respiratory viruses may induce host metabolite alterations by infecting epithelial cells. Liquid chromatography quadrupole time-of-flight mass spectrometry with machine learning was evaluated to identify distinct metabolic signatures from nasopharyngeal samples for influenza diagnosis. A total of 236 samples were tested in the discovery phase, and analysis showed an area under the receiver operating characteristic curve (AUC) of 1.00 (95% CI 0.99, 1.00), sensitivity of 1.00 (95% CI 0.86, 1.00) and specificity of 0.96 (95% CI 0.81, 0.99). Prospective validation of a 20-biomarker signature optimized for sensitivity in 96 individuals revealed an AUC of 0.99 (95% CI 0.97, 1.00), sensitivity of 1.00 (95% CI 0.93, 1.00) and specificity of 0.69 (95% CI 0.55, 0.80). Therefore, it was discovered that this metabolomic approach is useful in infectious disease evaluations, including other diagnostics applications related to respiratory viruses such as severe acute respiratory syndrome coronavirus 2 (SARS-CoV-2). Further, the embodiments described herein are useful in point-of-care testing.

Over the last decade, the diagnosis and monitoring of infectious diseases has been revolutionized by molecular testing, including the widespread use of Polymerase Chain Reaction (PCR), in addition to other amplification, nucleic acid and protein detection techniques in Clinical Microbiology and Virology Laboratories. Many of these methods are rapid and highly accurate. However, important limitations remain unaddressed, including high cost, high complexity, inability to differentiate active infection from latency or colonization, and lack of sensitivity in direct patient specimens. Moreover, molecular testing is often restricted to high complexity laboratories, far from the point of care where prompt and actionable diagnosis is most needed. Accurate testing is particularly important for respiratory viruses including influenza, which are estimated to have caused over 35 million symptomatic illnesses during the 2018-2019 season alone in the United States. Such testing is also essential for the early diagnosis of severe acute respiratory syndrome coronavirus-2 (SARS-CoV-2) and other similar and analogous infectious diseases. These viruses infect respiratory epithelial cells, where they may induce metabolite alterations in the host. The ‘-omics’ field has emerged as a promising discipline to address some of these gaps, with greater emphasis placed on genomics and proteomics so far for infectious diseases diagnostics including clinical virology. Metabolomics, or the large-scale study of small molecules, represents a change in paradigm from routine clinical virology diagnostics as it detects host metabolic response rather than directly detecting pathogen. Metabolomics, including the embodiments described herein, provides significant and unforeseen utility in infectious diseases applications as it can be performed directly from patient specimens, is inexpensive to run, and may accurately differentiate active infection from colonization.

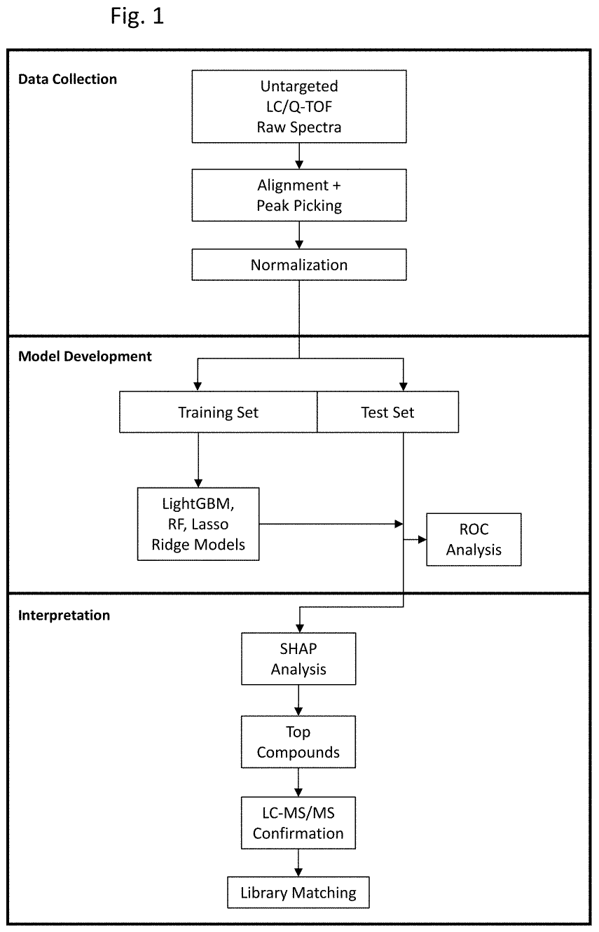

Nasopharyngeal swab sampling followed by swab immersion in viral transport medium (VTM) is the most common collection technique for the diagnosis of respiratory viruses and enables the non-invasive collection of respiratory cells. The inventors have determined that analysis of VTM after nasopharyngeal sampling using a recently reported and sensitive metabolomics method reveals distinct metabolomics signatures for the diagnosis of infectious diseases. The present exemplary methods are based on an in-line, two-column chromatographic arrangement that allows the capture of both non-polar and polar compounds in a single (e.g., 20-minute) run, and is well suited for the characterization of host metabolite signatures directly from patient specimens using liquid chromatography quadrupole time-of-flight mass spectrometry (LC/Q-TOF). In the present example, a LC/Q-TOF method was utilized to generate data to develop and validate machine learning (ML) algorithms for classification of influenza infection status, and an interpretation method for biomarker discovery (FIG. 1).

A total of 248 samples were obtained and subjected to the methods described herein. After processing, the samples were analyzed by LC/Q-TOF for metabolite discovery. Of these, 6 were excluded prior to analysis due to technical errors and their 6 corresponding controls were excluded. The final analysis included a total of 236 samples, with 118 positive influenza samples (40 influenza A 2009 H1N1, 39 influenza A H3 and 39 influenza B) and 118 negative age and sex-matched controls (Table 1). Compared to individuals without influenza, those with a positive influenza result were more likely to have been tested at an outpatient clinic (63.6% vs 26.3%; p<0.001), less likely to be immunocompromised (22.9% vs 45.8%; p=0.001), less likely to have been hospitalized (24.6% vs 69.5%; p<0.001) and less likely to have been admitted to the intensive care unit (ICU) (5.1% vs 22.0%; p<0.001). Patient characteristics were otherwise similar. All-cause 30-day mortality was identical in each group at 3/118 (2.5%).

TABLE 1
Baseline demographic characteristics of all patients
in the untargeted metabolomics phase of the study

| | | Negative for | | Positive for any | | |
|---|---|---|---|---|---|---|
| | | respiratory | | respiratory virus | | |
| | | viruses (n = 118) | | (n = 118) | | p-value* |
| Age | ≥2yo-17yo | 48 | (40.7%) | 48 | (40.7%) | 1.0 |
| | ≥18yo | 70 | (59.3%) | 70 | (59.3%) | |
| Sex | Male | 62 | (52.5) | 61 | (51.7) | 0.9 |
| | Female | 56 | (47.5) | 57 | (48.3) | |
| Immunocompromised | Yes | 54 | (45.8%) | 27 | (22.9%) | 0.001 |
| | No | 63 | (53.4%) | 87 | (73.7%) | |
| | Unknown | 1 | (0.8%) | 4 | (3.4%) | |
| Comorbidities | Leukemia/lymphoma | 27 | (22.9%) | 10 | (8.5%) | 0.005 |
| | Active malignancy | 10 | (8.5%) | 2 | (1.7%) | 0.02 |
| | Asthma | 6 | (5.1%) | 7 | (5.9%) | 0.5 |
| Charlson comorbidity index score | | 1 | (0-3) | 0 | (0-2) | 0.002 |
| (median; IQR) | | | | | | |
| Days of symptoms at the time of testing | | 3 | (1-7) | 3 | (2-9) | 0.4 |
| (mean; SD) | | | | | | |
| Patient location | ED | 41 | (34.8%) | 36 | (30.5%) | <0.001 |
| | ICU | 16 | (13.6%) | 4 | (3.4%) | |
| | Inpatient ward | 30 | (25.4%) | 3 | (2.5%) | |
| | Outpatient clinic | 31 | (26.3%) | 75 | (63.6%) | |
| Antiviral treatment at | Yes | 0 | | 3 | (2.5%) | 0.1 |
| time of testing | | | | | | |
| | No | 114 | (96.6%) | 96 | (81.4%) | |
| | Unknown | 4 | (3.4%) | 19 | (16.1%) | |
| Hospitalization | Yes | 82 | (69.5%) | 29 | (24.6%) | <0.001 |
| | No | 36 | (30.5%) | 89 | (75.4%) | |
| ICU admission | Yes | 26 | (22.0%) | 6 | (5.1%) | <0.001 |
| | No | 92 | (78.0%) | 112 | (94.9%) | |
| 30-day all-cause | Yes | 3 | (2.5%) | 3 | (2.5%) | 1.0 |
| mortality | | | | | | |
| | No | 115 | (97.5%) | 116 | (97.5%) | |
| | Inpatient ward | 30 | (25.4%) | 3 | (2.5%) | |
| | Outpatient clinic | 31 | (26.3%) | 75 | (63.6%) | |
| Antiviral treatment | Yes | 0 | | 3 | (2.5%) | 0.1 |
| at time of testing | | | | | | |
| | No | 114 | (96.6%) | 96 | (81.4%) | |
| | Unknown | 4 | (3.4%) | 19 | (16.1%) | |
| Hospitalization | Yes | 82 | (69.5%) | 29 | (24.6%) | <0.001 |
| | No | 36 | (30.5%) | 89 | (75.4%) | |
| ICU admission | Yes | 26 | (22.0%) | 6 | (5.1%) | <0.001 |
| | No | 92 | (78.0%) | 112 | (94.9%) | |
| 30-day all-cause | Yes | 3 | (2.5%) | 3 | (2.5%) | 1.0 |
| mortality | No | 115 | (97.5%) | 116 | (97.5%) | |

The p values were calculated by Chi-squared if categorical variables, by Fisher's exact test if categorical variables with less than 5 datapoints per cell, and by Mann Whitney U test for continuous variables. ED: emergency department; ICU: intensive care unit; IQR: inter-quartile range; SD: standard deviation; yo: years-old.

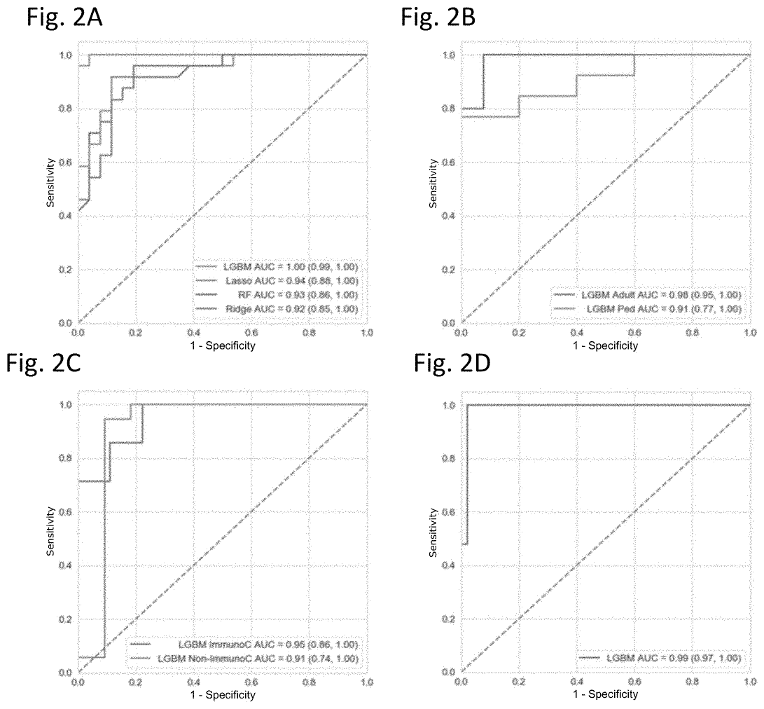

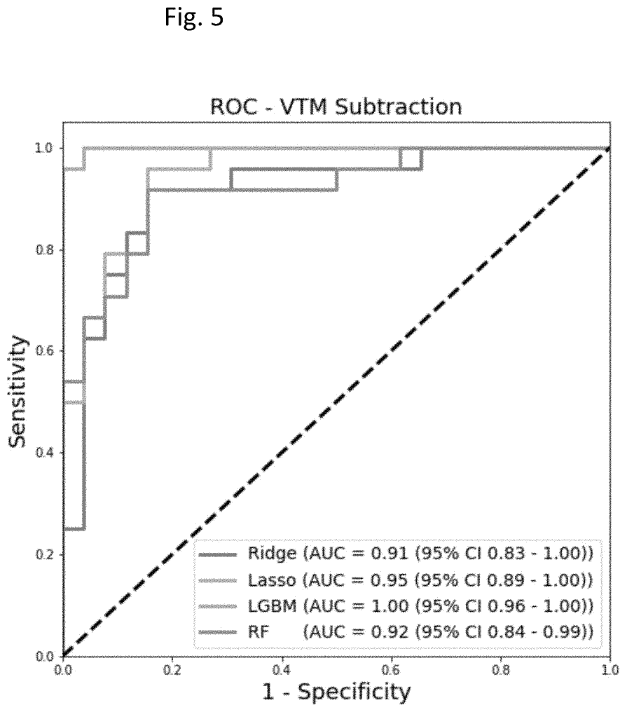

Untargeted metabolomics machine learning results show high classification performance: The discovery cohort training set consisted of 186 samples (94 positive, 92 negative), and the test set consisted of 50 samples (24 positive, 26 negative). Untargeted metabolomics identified a total of 3,366 ion features. Of these, 48 ion features were removed since they showed “zero” values for all samples tested, leaving 3,318 ion features for analysis. Application of machine learning models to these features, specifically the LightGBM (LGBM) and random forest (RF) models, achieved an area under the receiver operating characteristic curve (AUC) of 1.00 (95% CI 0.99, 1.00) and 0.93 (95% CI 0.86, 1.00) respectively on the test set. Statistical models, specifically the Lasso and Ridge regression models, obtained AUCs of 0.94 (95% CI 0.88, 1.00) and 0.92 (95% CI 0.85, 1.00) respectively. Subtraction of the background spectral data from the blank VTM sample replicates did not impact test performance of the model (FIG. 5). At an operating point optimized for sensitivity, LGBM achieved a sensitivity of 1.00 (95% CI 0.86, 1.00) and a specificity of 0.96 (95% CI 0.81, 0.99), superior to other models (Table 51). Subgroup analysis of the performance of the LGBM model on adults and children showed an AUC of 0.98 (95% CI 0.95, 1.00) for adults and an AUC of 0.91 (95% CI 0.77, 1.00) for children (FIG. 2B). The same model demonstrated an AUC of 0.95 (95% CI 0.86, 1.00) in immunocompromised hosts, and an AUC of 0.91 (95% CI 0.74, 1.00) in non-immunocompromised hosts (FIG. 2C). Only 32 individuals in this cohort were hospitalized to the intensive care unit (ICU); AUC was 1.00 (95% CI 0, 1.00) in ICU patients compared to AUC 0.94 (95% CI 0.85, 1.00) in non-ICU patients. Data from the other models are presented in Table S2.

TABLE S1
Sensitivity and specificity values for machine learning and
statistical models (LGBM: LightGBM; RF: random forests)

| | Ridge | Lasso | LGBM | | RF |
|---|---|---|---|---|---|
| Sensitivity | 0.92 (0.74-0.98) | 0.88 (0.69-0.96) | 1 | (0.86-1) | 0.79 (0.60-0.91) |
| Specificity | 0.81 (0.62-0.91) | 0.88 (0.71-0.96) | 0.96 | (0.81-0.99) | 0.88 (0.71-0.96) |

TABLE S2
Subgroup analyses for AUC data for adult vs pediatrics, immunocompromised vs non-
immunocompromised individuals, and ICU-admitted vs non-ICU-admitted individuals (IC:
Immunocompromised; ICU: intensive care unit; LGBM: LightGBM; RF: random forests)

| Subgroups | | Ridge | | Lasso | | LGBM | | RF | |
|---|---|---|---|---|---|---|---|---|---|
| Age | Adult | 0.90 | (0.78-1) | 0.85 | (0.70-0.99) | 0.98 | (0.95-1) | 0.95 | (0.89-1) |
| | Pediatric | 0.85 | (0.66-1) | 0.85 | (0.66-1) | 0.91 | (0.77-1) | 0.88 | (0.71-1) |
| Severity | ICU | 1 | (0-1) | 0.75 | (0-1) | 1 | (0-1) | 1 | (0-1) |
| | Non-ICU | 0.86 | (0.74-0.99) | 0.94 | (0.87-1) | 0.94 | (0.85-1) | 0.89 | (0.77-1) |
| Host | IC | 0.92 | (0.78-1) | 0.92 | (0.78-1) | 0.95 | (0.86-1) | 0.86 | (0.67-1) |
| status | Non-IC | 0.91 | (0.80-1) | 0.97 | (0.91-1) | 0.91 | (0.74-1) | 0.81 | (0.61-1) |

Application of a parsimonious biomarker signature maintains high performance

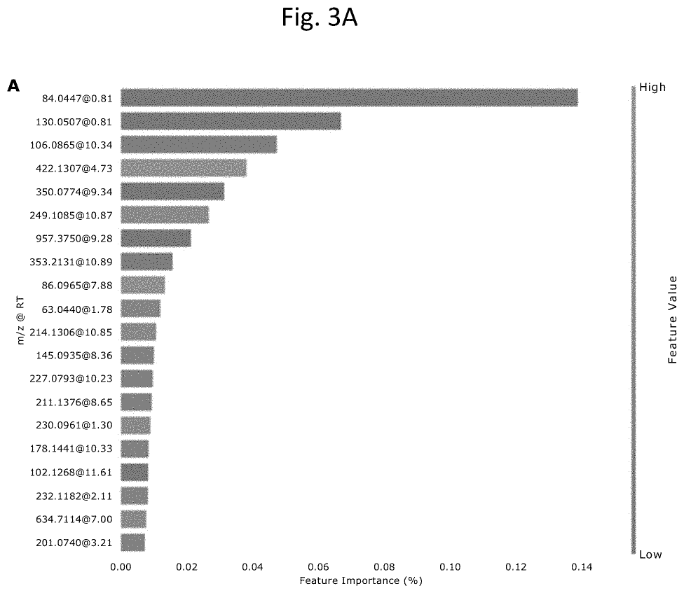

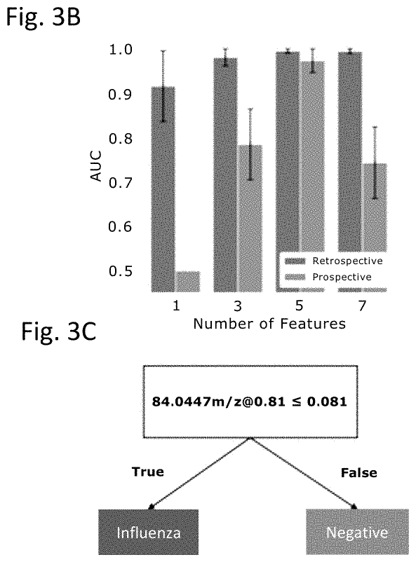

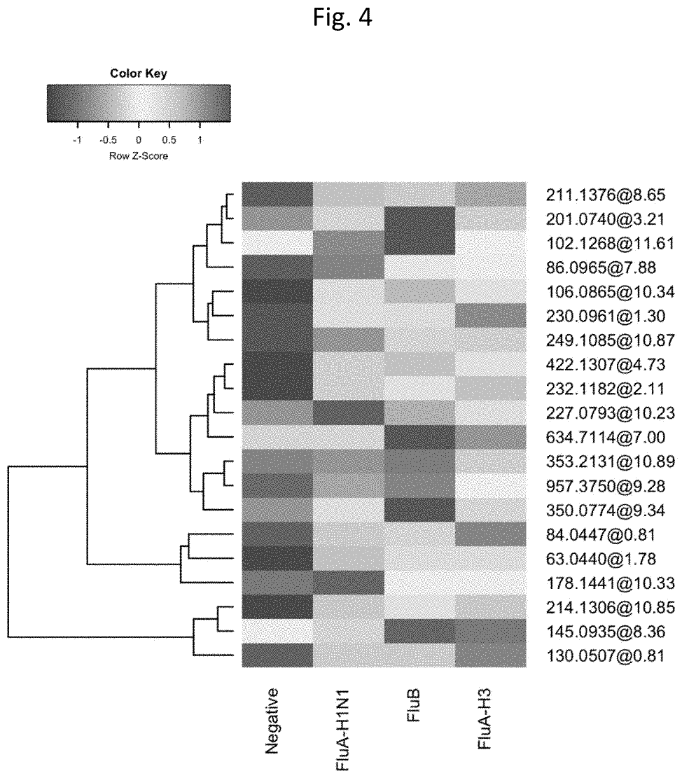

After ranking features by importance, the top 20 ion features associated with classification were identified, of which only 13 contributed more than 1% to model predictions (FIGS. 3A-3C, 4). This top 20 biomarker signature was validated in a prospective cohort of 96 symptomatic individuals with nasopharyngeal swabs including 48 positives (24 influenza A H1N1, 5 influenza A H3 and 19 influenza B) and 48 negatives. This signature revealed an AUC of 0.99 (95% CI 0.97, 1.00), sensitivity of 1.00 (95% CI 0.93, 1.00) and specificity of 0.69 (95% CI 0.55, 0.80) (FIG. 2d). Decision tree models trained using the top 3, 5, and 7 features obtained AUCs of 0.98 (95% CI 0.96, 1.00), 1.00 (95% CI 0.99, 1.00), and 0.99 (95% CI 0.99, 1.00), respectively (FIG. 2b).

Thus, use of a decision tree model trained on only the top 5 features achieved performance comparable to the LGBM model on the full feature set. In addition, building a classifier using a single decision on the top feature achieved an AUC of greater than 0.9 on the holdout test set (FIG. 2c). In the prospective cohort, models trained using the top 3, 5, and 7 features obtained AUCs of 0.78 (95% CI 0.70, 0.86), 0.97 (95% CI 0.94, 1.00) and 0.74 (95% CI 0.66, 0.82), respectively (FIG. 2b).

Pyroglutamic acid identified as top metabolite: We conducted metabolite identification through library matching to reveal a Tier 1 match for compound 130.0507@0.81 as pyroglutamic acid, and compound 84.0447@0.81 as an in-source fragment ion of pyroglutamic acid. Further metabolite annotation work will identify the chemical entities comprising the other metabolites listed.

Molecular testing has revolutionized the diagnosis of respiratory viral infections in clinical laboratories, with multiplexed reverse transcriptase polymerase chain reaction (RT-PCR) representing the current standard of care. However, limitations to this technique remain, including high cost and the inability to differentiate active infection from persistent nucleic acid, thus improved diagnostic tools are needed. In addition, the target-specific approach of multiplexed panels has revealed its shortcomings in its inability to diagnose emerging viruses such as severe acute respiratory syndrome coronavirus 2 (SARS-CoV-2). Furthermore, the high complexity of many molecular assays limits their use at the point of care where the patient need for a rapid and actionable diagnosis is highest. Metabolomics, or the large-scale study of small molecules, represents the ‘-omics’ technology closest to phenotype and thus holds promise to address current gaps in molecular testing of infectious diseases. This is particularly important given the significant burden of respiratory viruses in the U.S. and internationally, and the ongoing major unmet need to expand diagnostic testing modalities for the early diagnosis of SARS-CoV-2, the causative agent of coronavirus disease (COVID-19). For example, the nature of a certain infectious disease that is not yet identified or identifiable through a rapid test or commercially available testing can be identified very early into its emergence in patients using the methods of the present disclosure.

In an exemplary study described herein 236 nasopharyngeal swab samples from symptomatic individuals were obtained, processes and evaluated. It is shown herein that the described LC/Q-TOF methods, optionally combined with machine learning, can differentiate between influenza-positive (including influenza A 2009 H1N1, H3 and influenza B) and influenza-negative samples with high test performance including sensitivity, specificity and AUC over 0.90. Given the novelty of this approach, comparative datapoints for this application are lacking. However, this approach compared favorably to a previous study using an unbiased proteomic strategy from nasopharyngeal lavage sampling with normal saline from 15 previously healthy hosts experimentally infected with influenza A H3N2 or human rhinovirus. T. W. Burke et al., Nasopharyngeal Protein Biomarkers of Acute Respiratory Virus Infection. EBioMedicine 17, 172-181 (2017). The 10-peptide signature from that study was validated in a cohort of 80 subjects, achieving overall AUC of 0.86, sensitivity of 75% and specificity of 97.5% including paired samples.

The metabolomics sample processing presented here is simpler and faster than proteomic workflow (approximately 30 minutes for ultrafiltration compared to >20 hours for proteomics), thus conferring a distinct advantage even at similar performance.

The top 20 differentially expressed ion features retained in our biomarker signature likely represent a heterogeneous group of compounds from a variety of biological pathways. As noted, the top two ion features were identified as pyroglutamic acid (130.0507@0.81) and an in-source fragment ion of pyroglutamic acid (compound 84.0447@0.81), which are decreased in specimens from influenza-infected individuals. Pyroglutamic acid (synonyms: pidolic acid, 5-oxoproline) is a cyclized derivative of L-glutamic acid which can form in one of three ways in the living cell; from the degradation of glutathione, from incomplete reactions following glutamate activation, or from the degradation of proteins containing pyroglutamic acid at the N-terminus. The present results show a decrease in pyroglutamic acid in NP swabs from influenza-infected individuals. Given the utilized samples are not washed or lysed, the observed decrease in pyroglutamic acid in NP swabs from infected individuals may be due to decreased extracellular concentrations from increased use of glutathione in the intracellular space. Alternatively, while not intending to be bound to any particular theory, a more complex mechanism involving oxidative stress and upstream metabolic effects may be at play. Though the mechanism giving rise to differential concentrations of pyroglutamic acid in our specimens is not yet known, our results further suggest and highlight glutathione metabolism as a key pathway altered during influenza infection.

In the present example, statistical models and machine learning models were utilized to assess for best test performance for untargeted metabolomics data. The inventors found the results to be reproducible across datasets and across models, adding confidence to the findings. Furthermore, the machine learning models were observed to consistently outperform the statistical models.

This present example demonstrates multiple strengths. First, it demonstrated high test performance in the discovery cohort, which was independently validated in a prospective cohort of consecutive individuals, supporting the reproducibility and robustness of this approach. Second, it demonstrated a large effect size from a limited number of compounds in the SHAP feature importance analysis. This increases the feasibility of adapting this diagnostic approach to a point-of-care device such as portable mass spectrometry. Third, this study was based on a real-world, diverse patient population of individuals who were naturally infected with influenza, which may better approximate metabolic changes compared to experimentally-infected, previously healthy volunteers. Furthermore, cases and controls in the discovery cohort were tightly age- and sex-matched, thus reducing potential confounders in metabolomic analysis due to up- or downregulation of certain metabolic pathways based on these host factors. Fourth, this cohort included a large number of samples, conferring over 90% power to detect a difference between influenza-infected and uninfected individuals. Finally, herein provided is a systematic and comprehensive bioinformatics pipeline analysis strategy to identify the best model for untargeted and targeted metabolomics data.

Further, herein the feasibility and high accuracy of the presently described metabolomics approach from nasopharyngeal samples for the identification of distinct metabolic signatures for the diagnosis of influenza infection is presented. This approach required simple sample processing, low sample volume and was inexpensive to run. Testing in other patient settings, additional pathogens and sample types, will confirm and expand these results and further support the claimed embodiments as prognostic and/or diagnostic tools. This approach is useful, for example, for or in the diagnosis of COVID-19. In addition, is it contemplated that the methods described herein are used to explore metabolic pathways that could eventually be harnessed for therapeutic potential.

Material and Methods: The research objective was to assess the diagnostic test performance of the LC/Q-TOF (discovery cohort) and targeted analysis (prospective cohort) for the diagnosis of influenza-infected vs. uninfected individuals, and to identify key metabolites for classification of these two groups. In both the discovery and prospective cohorts, target sample size was determined before the experiments to achieve over 90% power based on an AUC of 0.925 for detection of a difference in the primary outcome of influenza infection vs. no infection. A secondary endpoint of influenza A vs. influenza B was established in the study design phase and used as an exploratory endpoint. The target sample was not changed during the study. Nasopharyngeal samples collected from adult patients were processed per routine clinical procedures. Briefly, a flocked swab is inserted in the nasal passage, rotated for collection of cells for 10-15 seconds and placed in viral transport medium (MicroTest M4RT, Remel Inc., San Diego, Calif.). Respiratory viral testing was performed on the ePlex Respiratory Pathogen (RP) panel (GenMark Diagnostics, Carlsbad, Calif.) at the Stanford Clinical Virology Laboratory. This automated qualitative nucleic acid amplification test (NAAT) identifies 15 viral targets, including influenza A, influenza H1N1 2009, influenza A H3 and influenza B. Specimens were aliquoted and stored at −80° C. for subsequent LC/Q-TOF testing.

For the discovery cohort, stored specimens collected from a specific time duration were utilized to achieve a 1:1 ratio of positive to age and sex-matched negative controls if possible. Age-matching was performed to the identical age, or within 2 years if not available. Specimens from 96 children (2-17 years-old) and 140 adults (18 years-old) were included. These corresponded to 123 males and 113 females. Mixed infections and samples from other sites (e.g., oropharyngeal swab, bronchoalveolar lavage and lung tissue) were excluded. Individual retrospective chart review was performed for all subjects in the untargeted phase of the study to identify age, sex, immunocompromised status, comorbidities, disease severity, antiviral treatment and clinical outcomes. LC/Q-TOF testing was performed to generate raw data on mass-to-charge ratio and retention time for each sample tested. Single replicate testing was performed, and outlier data points were included for analysis. For the prospective cohort, we selected consecutive negative and positive nasopharyngeal swab specimens from a specific time duration in a 1:1 ratio without exclusion. We included specimens from 15 children and 81 adults, corresponding to a total of 40 females and 56 males. LC/MS-MS testing was performed to generate raw data on mass-to-charge ratio and retention time for each sample tested. Single replicate testing was performed, and outlier data points were included for analysis. This method served to confirm the results from the LC/Q-TOF analysis in a separate participant cohort.

The following LC-MS grade reagents were used for the experiments: methanol and formic acid (Fisher Scientific, Chino, Calif.), ammonium formate salt and high-purity ammonium hydroxide (25% v/v) (Sigma Aldrich, St. Louis, Mo.) and water (VWR, Visalia, Calif.). In addition, high-pressure liquid chromatography (HPLC) grade acetonitrile and isopropanol (VWR), and MS calibration and reference mass solutions (Agilent Technologies, Santa Clara, Calif.) were used. The Mass Spectrometry Metabolite Library of Standards was purchased to build the in-house reference database (IROA Technologies, Boston, Mass.), and was complemented by additional standards (Sigma-Aldrich).

Exemplary LC/Q-TOF Method: Liquid chromatography (LC) separation was performed on an Agilent 1290 Quaternary LC system (Agilent Technologies). In this unique chromatographic arrangement, two columns are used in-line: a reverse-phase (RP) column of 2.1×50 mm 1.8 μm HSS T3 (Waters Corporation, Milford, Mass.) is placed first followed by an ion exchange (IEX) column of 2.0×30 mm 3 μm Intrada (Imtakt USA, Portland, Oreg.). Both columns are joined with EXP2 fittings (Optimize Technologies, Oreg.). Mass spectrometry was performed on an Agilent 6545 Q-TOF instrument with electrospray ionization. The mobiles phases were A) 150 mg of ammonium formate per liter water with 0.4% formic acid (v/v), B) 1.2 g of ammonium formate per liter of methanol with 0.2% formic acid, and C) water with 1% each formic acid and ammonium hydroxide, as previously described. The flow rate was 0.5 mL/minute, column temperature of 45° C. and injection volume of 5 μL, for a total run time of 20 minutes (inject-to-inject). MS was performed on an Agilent 6545 Q-TOF with dual Agilent JetStream electrospray ionization, as previously described. The instrument was operated in sensitivity-mode with extended dynamic range and positive polarity, scanning from 50-1100 m/z.

LC/Q-TOF Metabolite extraction and analysis: A volume of 100 μL of nasopharyngeal sample eluted in VTM was processed by ultrafiltration using Pall Omega 3 kDa centrifugal devices (VWR, Radnor, Pa.) at 4° C. for 15 minutes at 17,000×g. The filtrate was transferred to glass vials and analyzed, and each sample was run once. Two quality controls (QC) samples, one pooled QC and an independent normalization QC were used to assess for batch effect. The pooled QC was created by pooling an equal volume of aliquots from all the samples included in the run. Unsupervised principal component analysis was performed to visually assess appropriate performance of the pooled QC. In addition, blank VTM was run in triplicate to generate a mean background spectral distribution. Progenesis QI software (Waters Corporation) was used for run alignment, peak picking (automatic, level 4), adduct deconvolution, and feature identification. Positive polarity analysis was performed using the adducts [M+H], [M+NH4] and [M+Na]. Metabolite identification was first performed using a previously-developed authentic standard library. If there was no identification match, preliminary annotation was performed in Progenesis QI software using the HMDB (33) and KEGG (34) plug-ins, and by manual review in METLIN. A mass error setting of 30 ppm was used. Data were directly exported from Progenesis for machine learning analysis using peak area filters of 0; 5,000; 10,000 and 20,000 relative abundance values. Outlier values were not excluded.

LC-MS/MS Targeted method: The targeted analysis was performed on a clinically-validated method that detects pyroglutamic acid. Mass spectrometry was performed on an Agilent 6460 Triple Quadrupole Mass Spectrometer equipped with an Agilent JetStream electrospray ionization, as described abobe. Additional selected reaction monitoring pairs based on the important ion features were added to the method (Table S3). Liquid chromatography separation was performed on a two-dimensional Agilent 1200 2× Binary LC system (Agilent Technologies), as described above. Two columns were connected using a 10-port switching valve (Rheodyne). First dimensional separation used a Thermo Hypercarb column, 3×50 mm, 3 μm (Thermo, UK). Second dimensional separation used a Waters BEH C18 column, 2.1×100 mm, 2.5 μm (Waters Corporation). Mobile phase A, 0.03% perfluoroheptanoic acid in water, is identical for both pumps 1 and 2. Mobile phase B, acetonitrile, is identical for both pumps 1 and 2. The data were acquired using MassHunter WorkStation Acquisition version B.08.02 (Agilent) and exported for ML analysis.

TABLE S3
Selected multiple reaction monitoring (SRM) pairs added to the LC/MS-MS Analysis
Compounds are listed by name or by accurate mass @ retention time.

| | | | Retention | | Collision |
|---|---|---|---|---|---|
| | | | Time | | Energy |
| Compound | Q1 | Q3 | (min) | Fragmentor | (volts) |
| Pyroglutamic Acid | 130.1 | 84 | 1.8 | 76 | 13 |
| Pyroglutamic Acid | 130.1 | 56.2 | 1.7 | 76 | 29 |
| Pyroglutamic Acid-D5 | 135.08 | 89.1 | 1.8 | 88 | 13 |
| Pyroglutamic Acid-D5 | 135.08 | 61.2 | 1.8 | 88 | 25 |
| 106.0865@10.34 | 106.1 | 58.2 | 5.26 | 100 | 15 |
| 145.0935@8.36 | 145.1 | 104 | 7.93 | 100 | 15 |
| 178.1441@10.33 | 178.1 | 119.2 | 7.39 | 100 | 15 |
| 201.0740@3.21 | 201.1 | 101.2 | 2.95 | 100 | 15 |
| 211.1376@8.65 | 211.1 | 70.2 | 2.82 | 100 | 15 |
| 214.1306@10.85 | 214.1 | 155.2 | 4.92 | 100 | 15 |
| 227.0793@10.23 | 227.1 | 114.2 | 3.33 | 100 | 15 |
| 230.0961@1.30 | 230.1 | 109 | 10.16 | 100 | 15 |
| 232.1182@2.11 | 232.1 | 85.3 | 7.68 | 100 | 15 |
| 249.1085@10.87 | 249.1 | 114.2 | 5.32 | 100 | 15 |
| 350.0774@9.34 | 350.1 | 220.9 | 3.2 | 100 | 15 |
| 350.0774@9.34 | 350.1 | 180.1 | 3.2 | 100 | 15 |
| 353.2131@10.89 | 353.2 | 160.1 | 6.67 | 100 | 15 |
| 422.1307@4.73 | 422.1 | 143 | 5.1 | 100 | 15 |
| 63.0440@1.78 | 63.0 | 45.2 | 4.72 | 100 | 15 |
| 634.7114@7.00 | 634.7 | 593.7 | 9.85 | 100 | 15 |
| 634.7114@7.00 | 634.7 | 552.7 | 9.85 | 100 | 15 |
| 84.0447@0.81 | 84.0 | 56.2 | 3.33 | 100 | 15 |
| 86.0965@7.88 | 86.1 | 69.2 | 2.14 | 100 | 15 |
| 957.3750@9.28 | 957.4 | 571.3 | 4.19 | 100 | 15 |
| 102.1268@11.61 | 102.1 | 56.1 | 2.33 | 100 | 15 |

LC-MS/MS Metabolite extraction and analysis: A volume of 100 μL of nasopharyngeal sample eluted in VTM and 10 μL of pyroglutamic acid-D5 as internal standard (Cambridge Isotope Laboratories, Inc, Tewksbury, Mass.) was processed by ultrafiltration using Pall Omega 3 kDa centrifugal devices (VWR, Radnor, Pa.) at 4° C. for 15 minutes at 17,000×g. The filtrate was transferred to glass vials and analyzed. MassHunter WorkStation Quantitative Analysis version B.07.00 (Agilent) was used for peak integration and data export for ML analysis.

Descriptive analysis was performed by Chi-squared test (categorical variables if 5 or more variables per cell) or Fisher's exact test (categorical variables if less than 5 variables per cell) and Mann-Whitney U test (continuous variables), using Stata v15.1 (Stata Corp, College Station, Tex.). Missing data are identified as unknown. A two-sided p value of <0.05 was considered significant.

Machine Learning Analysis: We developed and provide herein machine learning methods for the task of determining whether a sample was positive or negative for influenza based on its metabolic profile. Machine learning is a class of techniques that uses data to learn a model that maps an input (the metabolic profile of a sample; includes mass-to-charge ratio (m/z) and retention time for each sample) to its associated output (the influenza infection outcome of the sample) and uses this learned model on new inputs (the metabolic profiles of new samples) to make predictions of new outputs (the influenza outcomes of new samples). We implemented two machine learning methods: gradient boosted decision trees and RF.

Gradient boosted decision trees and RFs are both ensemble learning methods that improve upon the performance of decision tree models. Decision tree learners construct a model by iteratively identifying which feature most effectively divides the data into groups with low within-group variation in the outcome and high between-group variation in outcome, and then repeat the process within each group. Gradient boosted decision trees (GBDT) construct several decision trees such that each tree learns from the errors of the prior tree. Random forests construct several decision trees such that each tree is constructed using different subsets of the data. The machine learning approaches of GBDT and RF were chosen over alternative machine learning methods because they were discovered to be capable of handling mixes of categorical and continuous covariates, capture nonlinear relationships, and scale well to large amounts of data.

Dataset Splitting: As noted, ion features showing zero values through all samples tested were removed from the dataset. The remaining dataset was partitioned without normalization into a training set used to develop machine learning models, and a holdout test set used to evaluate the predictive performance of the machine learning models. The partitioning of the dataset was random such that 80% of the samples were included in the training set, and the other 20% in the test set. There was no overlap between the samples and patients between the two sets.

All models were developed on the training set, and their final performance reported on the holdout test set and/or the prospective cohort. Within the training set, cross-validation was used to develop the models to avoid overfitting to the training set. In the cross-validation procedure, the training dataset was randomly partitioned into k=4 equal sized subsamples consisting of an approximately equal percentage of each class. Of the k subsamples, a single subsample was retained as the validation data for the model, and the remaining k−1 subsamples were used to train a model. The cross-validation process was then repeated k times, with each of the k subsamples used exactly once as the validation data. Grid search was used to find the best set of hyperparameters for model training; the same hyperparameter settings were used across all k folds. The resulting k models (one from each fold) were used to make k sets of predictions on the test set, which were then averaged using a simple mean to make the final prediction for each sample in the test set.

Machine Learning Methods vs Traditional Linear Models: To determine the usefulness of capturing non-linear relationships with machine learning models, the modelling approaches using two machine learning methods, gradient boosted decision trees and random forests, were compared with two traditional linear models, Least absolute shrinkage and selection operator (Lasso) and Ridge. These models are variants of Logistic regression, a statistical model that uses the logistic function to model the outcome assuming a linear relationship between the features and the outcome. Lasso makes the same linear assumption but alters the model fitting process to select only a subset of the features for use in the final model rather than using all of them. Unlike Lasso, Ridge will not result in a sparse model, but rather addresses multicollinearity in the features by shrinking the weights assigned to correlated variables. The training and test sets, and the cross-validation strategy were identical across the machine learning models and traditional linear models.

The SHapley Additive exPlanations (SHAP) method was used to quantify the impact of each feature on the models. The method explains prediction by allocating credit among the input features; feature credit is calculated using SHAP Values as the change in the expected value of the model's prediction of improvement for a symptom when a feature is observed versus unknown. To uncover clinically important ion features that were globally predictive of the outcome, the SHAP values for the top 20 ion features on individual predictions were aggregated and reported along with their averaged absolute Shapley contributions as a percent of the contributions of all the features.

Parsimonious Model: We developed a set of parsimonious models that were designed to use a small subset of features identified to be important by the feature importance method. The top k features with highest overall importance to the machine learning models were used; we used k values of 1, 3, 5, and 7. On each of these choices, a single decision tree model was trained using the previously described cross-validation strategy to build the parsimonious model. Maximum depth was restricted to k, and we optimized additional hyperparameters using grid search during cross-validation. We compared the performance of the parsimonious models to the full models. Classification performance on the prospective cohort was evaluated using the models trained on the restricted feature set from the discovery cohort without modification. The validation data were not used to assess or refine the model tested in the prospective cohort.

Statistical Methods: The primary measure of model performance was the area under the receiver operating characteristic curve (AUC), which illustrates the diagnostic discriminative performance of the models. Performance measures for the models also included sensitivity, specificity, and accuracy at a high-sensitivity operating point used to binarize the model predictions. The high-sensitivity operating point was selected by selecting a high-sensitivity operating point on each of the k validation folds and averaging them: on each validation fold, an operating point that maximized the Youden's J statistic and produced a sensitivity of at least 0.9 was selected. To assess the variability in estimates, we provide 95% Wilson score confidence intervals for sensitivity, specificity, and accuracy and 95% DeLong confidence intervals for AUC.

Analyses were performed in Python version 3.6.8, using the LightGBM v2.2.3 implementation for gradient boosted decision trees, scikit-learn v0.20.2 for RF, stratified k-fold cross-validation and grid search (41), SHAP (SHapley Additive exPlanations) v0.29.1 for computing feature importance, and R version 3.5.0 for statistical analysis.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

We claim:

1. A computer-implemented method comprising:
generating or receiving a plurality of metabolite feature data using a processed sample from a subject with an unknown or uncertain diagnosis or prognosis;
applying selective metabolite features to the plurality of metabolite feature data to create a new data output; and
generating a diagnostic or prognostic indication for the subject based on the new data output,
wherein the selective metabolite features are obtained by subjecting a plurality of corresponding metabolite feature data to a LightGBM machine learning model and a random forest (RF) machine learning model to generate classified corresponding metabolite feature data, the classified corresponding metabolite feature data comprising the plurality of corresponding metabolite feature data organized based on a ranking of a plurality of mass spectrometry identified features; and identifying a subset of the classified corresponding metabolite features as the selective metabolite features for a disorder using a SHapley Additive exPlanations (SHAP) method.

2. The method of claim 1, wherein each of the plurality of metabolite feature data is obtained using a patient sample having a known diagnostic or prognostic status.

3. The method of claim 1, wherein the processed sample is obtained from eluting and processing a raw subject sample by liquid chromatography, and wherein the plurality of metabolite feature data is obtained by subjecting the processed sample to mass spectroscopy.

4. The method of claim 3, wherein the liquid chromatography is two column in-line liquid chromatography comprising reverse phase and ion exchange chromatography.

5. The method of claim 3, wherein the eluting and processing comprises ultrafiltration of the raw subject sample, and wherein the raw subject sample comprises a nasopharyngeal swap in transport medium.

6. The method of claim 1, wherein the selective metabolite features comprises one or more features.

7. The method of claim 6, wherein the selective metabolite features comprises three or more features.

8. The method of claim 6, wherein pyroglutamic acid is one of the selective metabolite features and the diagnostic or prognostic indication relates to influenza or infection by a respiratory virus.

9. The method of claim 1, wherein the diagnostic or prognostic indication relates to an infectious disease state, a cancer state, graft rejection state, a blood disorder, a soft tissue disorder, or an autoimmune disease state.

10. The method of claim 1, wherein the method is conducted at the point-of-care of the subject.

11. The method of claim 3, wherein the method is conducted at the point-of-care of the subject and wherein the mass spectroscopy is conducted using a portable mass spectroscopy device.

12. The method of claim 1, wherein the generated diagnostic or prognostic indication for the subject based on the new data output is utilized in conjunction with clinical data in a diagnosis of or prognosis for the subject.

13. The method of claim 1, wherein the subject is identified as eligible for treatment based on the diagnostic or prognostic indication without associated genetic or molecular data obtained from a raw sample corresponding to the processed sample.

14. The method of claim 13, wherein the treatment comprises treatment for influenza, another infectious respiratory disease, cancer, graft rejection, a blood disorder, a soft tissue disorder, or autoimmune disease.

15. A method of processing a biological sample from a subject for metabolomics classification, comprising:
either eluting and processing the biological sample by liquid chromatography to create a processed sample and subjecting the biological sample to mass spectrometry to obtain a plurality of metabolite feature data, or obtaining the plurality of metabolite feature data from a preprocessed sample;
subjecting the plurality of metabolite feature data to a LightGBM machine learning model and a random forest (RF) machine learning model to generate classified metabolite feature data, the classified metabolite feature data comprising the plurality of metabolite feature data organized based on a ranking of a plurality of mass spectrometry identified features; and
identifying a subset of the classified metabolite features as selective metabolite features for a disorder using a SHapley Additive exPlanations method.

16. The method of claim 15, wherein the classified metabolite features are applied to a sample or series of samples, including an agent-treated sample or samples, in a process of biomarker discovery or analysis.

17. A device adapted to conduct the method of claim 3.

18. The device of claim 17, wherein the device comprises a processor and is operably connected with computer executable code, memory and data storage to support the method in an onboard computer or a remote computer.

19. A method of processing a biological sample from a subject for metabolomics classification, comprising:
optionally subjecting the biological sample to mass spectrometry to obtain a plurality of metabolite feature data;
subjecting the plurality of metabolite feature data to a LightGBM machine learning model and a random forest (RF) machine learning model to generate classified metabolite feature data, the classified metabolite feature data comprising the plurality of metabolite feature data organized based on a ranking of a plurality of mass spectrometry identified features; and
identifying a subset of the classified metabolite features as selective.

20. The method of claim 19, wherein the mass spectrometry comprises liquid chromatography quadrupole time-of-flight mass spectrometry.
